(19) United States
(12) Patent Application Publication
(10) Pub. No.: US 2008/0139872 A1
(43) Pub. Date: Jun. 12, 2008

(54) Pad for stimulating cellular regeneration in a patient

(75) Inventor: Mark Pasula, Palm Beach Gardens, FL (US)

Correspondence Address: Roger S. Thompson, Esq.;Cohen Pontani Lieberman & Pavane LLP, Suite 1210, 551 Fifth Avenue, New York, NY, 10176, US

(73) Assignee: Dynamic Research, LLC

(21) Appl. No.: 11/894,550

(22) Filed: Aug. 20, 2007

Related U.S. Application Data
(63) Continuation-in-part of application No. 10/159,264, filed on May 29, 2002, now Pat. No. 7,258,658.

(30) Foreign Application Priority Data
May 29, 2002 (PL) P347807

(51) Int. Cl. A61N 2/02

(52) U.S. Cl. 600/13

(57) ABSTRACT

A flexible mattress pad for treating a patient by maintaining the patient in a low-frequency, low-intensity magnetic field for an extended period. The frequency of the magnetic field is preferably in the range of from about 40 Hz to about 80 Hz. The intensity of the magnetic field is less than about 2 mT. The patient may preferably use a thermoregulator to select a temperature for treatment, preferably between the temperatures of 65° F. and 100° F. This temperature should be maintained at a uniform temperature over his body, and at a constant temperature over time. The thermoregulator controls heating elements to heat the patient if the patient's body temperature is too low, and controls cooling apparatus, such as fans, air conditioning, or cooling coils, for cooling his patient if the body temperature is too high. The flexible body is no more than six inched thick, preferably no more than two inches thick.

### CROSS REFERENCE TO RELATED APPLICATIONS

This application is a continuation-in-part of prior filed U.S. patent application Ser. No. 10/159,264, filed May 29, 2002, issued Aug. 21, 2007 as U.S. Pat. No. 7,258,658.

### BACKGROUND OF THE INVENTION

1. Field of the Invention

This invention is directed to a pad for treating patients and, more particularly, to a portable, flexible, pad useful for treating patients by facilitating the body's own healing processes to stimulate cellular regeneration and thereby speed up healing of ailments suffered by the patients.

2. Description of the Related Art

When injured, or ill, the human body tends to work to repair itself. In many cases, “bed rest” is all that is required to cure minor aches, pains and illnesses, from a twisted ankle to the flu. Even more serious illnesses may respond to sustained periods of rest and recuperation. The body's own recuperative powers are not always enough for more serious injuries, however, or fast enough for some purposes. For example, football players (professional and amateur) take a tremendous pounding one day (game day) each week, and lesser poundings on practice days. There would be a tremendous benefit in performance if the healing for normal wear, for football players, could be accelerated.

It would also benefit non-athletes as well, if they could accelerate the body's own healing powers and thereby shorten any recovery period for recuperation from illnesses, trauma or any other infirmity. It would be especially useful to find ways to improve the body's own healing functions without use of invasive techniques, such as surgery, drugs, herbs or other remedies.

The difficulty lies in the fact that the body's own restorative processes are not yet completely understood, which has led to the exploration of so-called non-traditional cures, such as holistic medicine, herbal treatments, acupuncture, etc. No such treatment, however, has yet been demonstrated to stimulate true cellular level regeneration, and speed recovery to any great extent.

The inventors have discovered that low-level (in the range of no more than about 2 milli-Teslas: <2 mT and preferably as low as 10 micro-Teslas: <10 μT, or lower) magnetic fields work to stimulate cellular regeneration, and that this regeneration is enhanced when the body is kept at a regulated and constant temperature.

Preliminary research indicates that such treatment is found to provide beneficial effects in speeding recuperation for bruises, strains, and the like, encountered by athletes, and may also assist in recuperation from other illnesses, for example, from bedsores, to nerve damage, to almost any illness afflicting the human body.

There have been prior attempts to utilize alternating electromagnetic fields to promote healing in the human body.

A device for treating hypothermia with a magnetic field was disclosed in U.S. Pat. No. 4,685,462. This device includes two spiral induction coils coupled with an RF generator through an automatic frequency tuning system comprising capacitors and induction coils. In this device, the coils are placed in a non-conducting cylinder which is an element of its casing. One of the coils is fed from the RF generator by a signal of a frequency tuned to the resonance frequency of the device, another by mutual induction. The device is used at a frequency of 2 MHz to 20 MHz, and is intended only to warm a patient who has been subjected to extreme cold, such as a diver submerged in near freezing water. It does not disclose use of magnetic fields to increase cellular regeneration.

Another device, disclosed in European Patent No. 0217011, for medium and high frequency electromagnetic field treatment, includes a pulse generator, a modulator, an amplifier, a band filter and a current regulation circuit. In this device, generated pulses are modulated by their duration or timing, and are amplified by means of power selectors. The amplified signal is applied to the patient through a filter by means of induction coils or electrodes. A signal from the patient is supplied to the device where a regulation system sets the value of rated current which excites the coils and electrodes.

A device for effecting the local, alternating electric and magnetic field of low frequency is disclosed in Polish patent document No. 171930. This device is provided with sensors of the three components of the terrestrial and local magnetic fields, connected with low-pass filters and a microcontroller at the output of which there is connected a pulse generator with three coils coupled thereto, incorporating an input multiplexer with inputs that are sensors and an output multiplexer. Those sensors are connected in parallel between the input multiplexer and the output multiplexer through low-pass filters. This device is used to determine the parameters of all components of the terrestrial and local magnetic fields and of the parameters of the electric field of the biological organism. It detects and signals the occurrence of cyclotronic resonance for specific ions which affect the biological processes of the organism, but is not itself intended for use in treating patients.

European patent document No. 0995463, describes a method and a device for the generation of an electromagnetic field which affects biological processes in living tissue, especially in the human body, by means of a pulsating magnetic field. An electric signal consisting of many discrete pulses having a frequency of from 1 Hz to 1000 Hz is generated and supplied to a device which then generates an electromagnetic field. Preferably, single pulses are grouped in pulse groups and the duration of each pulse group ranges from 0.25 second to 1.2 second. This device does not disclose the use of long-term exposure to a low-frequency magnetic signal for treating a patient.

The disadvantage of all those known devices is that they apply relatively high magnitude magnetic fields in the intensity range of from 1 mT to 10 mT, or they generate a magnetic field in one direction only. Another disadvantage of known devices is that they affect only a specific selected part of the human body through large values of magnetic induction causing excessively rapid stimulation of metabolic processes, while at the same time affecting healthy organs with unnecessarily strong, and therefore potentially harmful, magnetic fields.

Exposure of the human body to strong magnetic fields for extended periods of time is known to cause cellular damage, and so high-intensity magnetic field generating devices are not suitable for use over long periods, such as convalescence from serious long-term illnesses or injuries.

The invention disclosed in co-pending application Ser. No. 10/159,264, filed May 29, 2002, and scheduled to issue on Aug. 21, 2007 as U.S. Pat. No. 7,258,658, addressed these problems by providing a method and apparatus for stimulating cellular regeneration. The apparatus disclosed therein, however, was a fairly large structure, contained in a standard bed. It would be useful, therefore, if the same usability could be found in a smaller, more portable, apparatus.

### SUMMARY OF THE INVENTION

It is therefore an object of the invention to provide an improved method and device for improving cellular regeneration in a patient by exposing the patient to a low-frequency, low-intensity magnetic field for extended periods in a portable device.

It is a further object of the invention to provide an improved method and device for treating patients in which cellular regeneration is enhanced by maintaining the body heat of the patient at a substantially constant level during exposure to the low-frequency, low-intensity magnetic field in a portable device.

The invention is a flexible and portable pad for stimulating cellular regeneration in a body of a patient, including an electromagnetic field generator for generating a low-frequency, low-intensity magnetic field, and a support for maintaining the body of the patient in the magnetic field during treatment. The frequency of the magnetic field is preferably in the range of from about 40 Hz to about 80 Hz. The intensity of the magnetic field is less than about 2 mT, preferably less than 20 μT, and most preferably less than 10 μT. In addition, because it is preferred that the body of the patient be maintained at a constant temperature, a thermoregulator may be provided to maintain the patient's body at a constant selected temperature for treatment. The thermoregulator controls heating elements to heat the patient if the patient's body temperature is too low, and controls cooling apparatus, such as fans, air conditioning, or cooling coils in the support, for cooling the patient if the body temperature is too high. The pad is no more than six inches thick, and preferably between one-half inch and one inch thick.

The various features of novelty which characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings, wherein like reference numerals delineate similar elements throughout the several views:

FIG. 1 shows a device in accordance with the invention, shown in exploded perspective,

FIG. 2 shows the device of FIG. 1 in the context of a system employing the inventive method,

FIG. 3 shows a perspective of an alternate embodiment of the frame of a device in accordance with the invention,

FIG. 4 shows a planar view of a coil used to generate a magnetic field in the devices of FIGS. 1-3,

FIG. 5 shows a plan view of an inventive mattress pad in accordance with the invention,

FIG. 6 shows a cross-section of the mattress pad of FIG. 5, taken along the line “A-A” thereof, and

FIG. 7 shows a cross-section of a detail of the mattress pad of FIG. 5, taken along the line “B-B” thereof.

### DETAILED DESCRIPTION OF THE PRESENTLY PREFERRED EMBODIMENTS

It is commonly known that electromagnetic fields having a frequency of from about 50 Hz to about 60 Hz and having a strength of about 4.0 kA/m, which corresponds to magnetic induction of approximately 5 mT, is not very good for human health. According to Polish safety regulations, a safe zone where humans may stay without time restraints is considered an area where the strength of a magnetic field of frequency 50 Hz is less than 0.4 kA/m, which corresponds to a magnetic induction of approximately 0.5 mT (500 μT). It is also known that when humans are exposed to magnetic fields less than about 70 μT, the magnetic field affects, through electromotive reactions, the mechanisms of physicochemical reactions of high selectivity by a respective broad-band lowering of the energy of cellular activation. In particular, the body's metabolic processes are stimulated in membrane areas.

We have discovered that, surprisingly, long-term exposure to low-intensity magnetic fields, having a low frequency, in the range of from about 50 Hz to about 60 Hz, lead to the stimulation of metabolic processes in mammals, including humans.

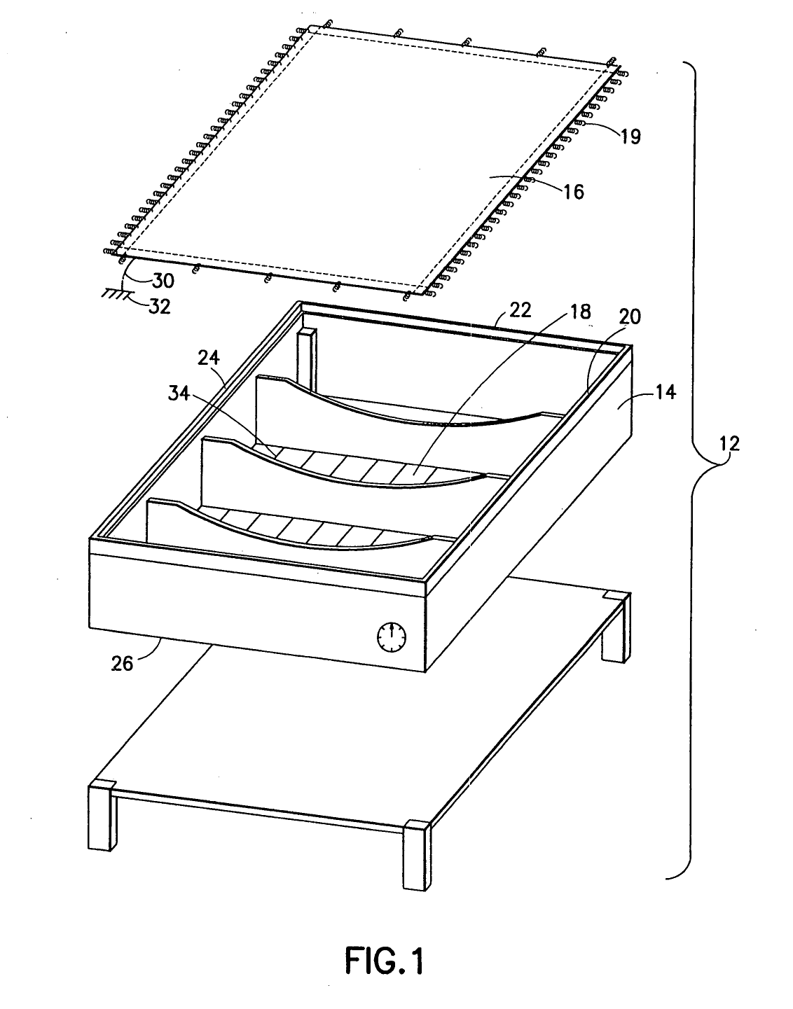

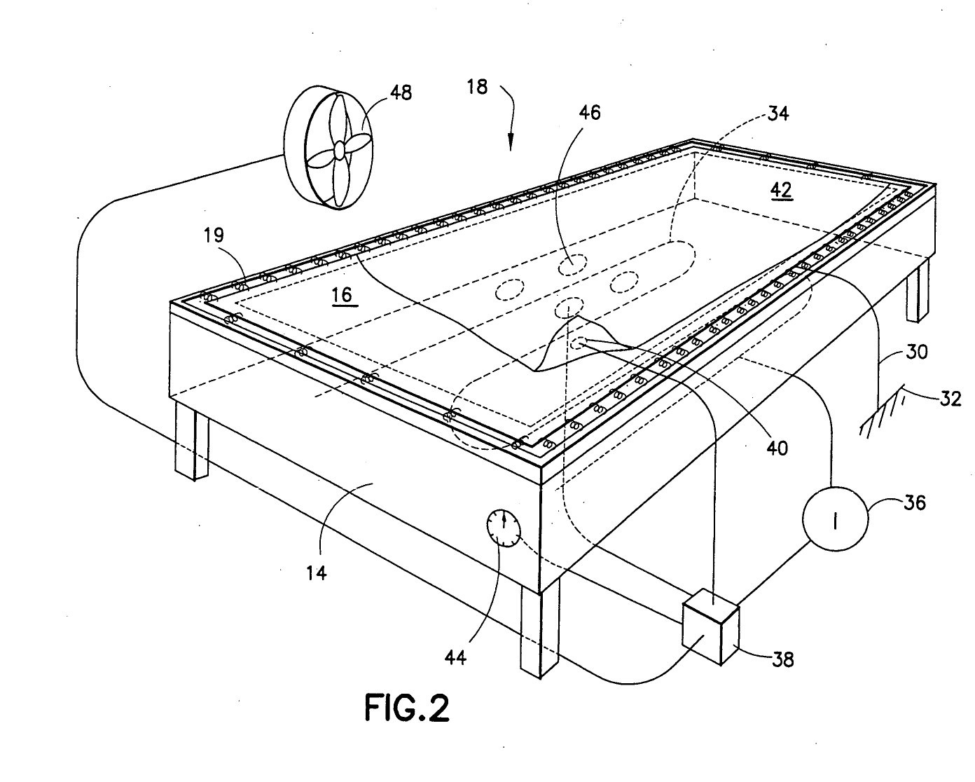

To facilitate the treatment of humans, or any other mammals with the use of low-frequency, low-intensity magnetic fields, we have constructed a system such as shown in FIGS. 1 and 2, which includes a bed 12, having a tight, preferably rectangular frame 14 which supports a resilient patient supporting surface 16. A means 18 for generating a magnetic field is disposed below supporting surface 16, so that the patient is disposed in the magnetic field generated by magnetic field generator 18, but preferably not in direct contact with generator 18. Often, portions of generator 18 may be hot, and so for the safety and comfort of the patient, direct contact therewith should be avoided.

Supporting surface 16 may be detachable and of any sort providing comfortable support to the patient, such as a conventional mattress, a thin fabric sheet which is preferably air permeable and suspended from frame 14 as by springs 19, or a specially adapted mattress in which the magnetic coils are disposed.

It is also possible supporting surface 16 may be eliminated and frame 14 is made to be water tight. Frame 14 is then filled with a fluid, such as water, as shown in FIG. 3, and the patient is partially immersed and suspended in the water, to avoid contact with a solid surface.

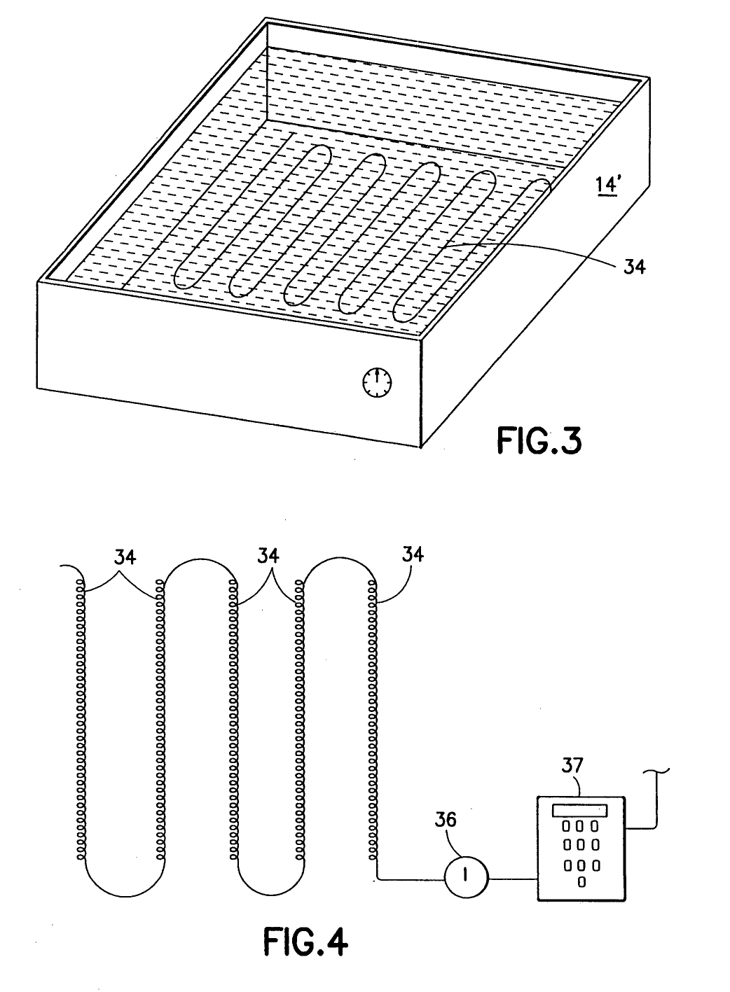

Frame 14 is preferably open at the top, and has four sides, 20, 22, 24 and 26. Sides 20, 22, 24 and 26 and supporting surface 16 are preferably lined with an insulating material to minimized heat loss. It is preferred that supporting surface 16 has a thin metal thread 30, most preferably made of copper, woven into it. Preferably, thread 30 should cover at least ⅔ of the length of the longer side of supporting surface 16 so that when the patient is laying on supporting surface 16, thread 30 is disposed proximate the patient, regardless of patient size. Thread 30 is connected to an electrical ground 32 and is used to carry excess electrostatic charge away from the patient. A set of coils 34 is disposed proximate supporting surface 16 and is selected to produce an electromagnetic field having a frequency in the range of from about 40 Hz to about 80 Hz, and preferably in the range of from about 50 Hz to about 60 Hz. Coils 34 are energized with alternating current of no more than 24 volts from a current source 36 (FIG. 4) in conventional fashion. The strength of the magnetic field is controlled by an adjuster 37 which, in response to input, alters the intensity of the current passing through coils 34 from current source 36. Preferably, coils 34 are arranged generally parallel to one another (except at their longitudinal ends, which are joined by U-shaped portions) proximate the entire support surface 16. Most preferably, coils 34 are placed on or between two fabrics or materials which are thermoconductive, allowing for the dissipation of heat therefrom. According to the preferred embodiment of the invention, the density of the turns of winding of individual coils is selected so that the value of the magnetic induction provided thereby is within the range of from about 1.2 μT to about 10 μT on the edges frame 14, and preferably should not exceed about 2.6 μT in the vicinity of the patient.

Turning to FIG. 2, to maintain the patient's body heat at a substantially constant selectable temperature, it is preferred that system 10 includes a thermoregulator 38, which comprises one or more conventional temperature sensors 40 placed in close proximity to the body of the patient, for example in supporting surface 16, in a cover 42 disposed on top of supporting surface 16, or on the body of the patient. A temperature selection means 44, such as a dial or digital input, is connected to thermoregulator 38 allow the patient or treating physician to select a comfortable or treating temperature. Preferably, the temperature is selected within the range of from 65° F. to about 100° F. The precise temperature is not believed critical, but it is believed most desirable that the patient be maintained at a substantially uniform temperature over his entire body, with the possible exception of the head, which is generally maintained outside of cover 42, for ease of breathing. This temperature should be substantially constant over time.

Sensors 40 transmit the value of the current body temperature of the patient to thermoregulator 38, which controls the temperature near the patient by switching an air chamber heating/cooling unit 46 on or off. Although four heating/cooling units 46 are shown, fewer or more units may be used. At the same time, coils 34 may be switched on or off to regulate the generation of the magnetic field from generator 18, to control heat generated thereby. It is preferred that the body temperature of the patient be maintained constant with a variance of no more than about 0.4° F. from the selected temperature. If the body temperature rises more than 0.4° F., the patient is cooled by heating/cooling units 46, while if the body temperature falls more than 0.4° F. below the desired temperature, the patient is warmed by heating/cooling units 46.

Heating may conveniently be accomplished by providing heating/cooling elements 46 in either or both of support surface 16 and cover 42, as, for example, by an electric blanket-type mechanism, as is well-known. Alternatively, external heating elements such as convection heaters, space heaters, hot air blowers, and the like may be provided. Cooling may likewise be accomplished in conventional fashion, by providing cooling coils in either or both of support surface 16 and cover 28, or by providing an ambient cooling element 48, such as an air conditioner, fan, ceiling fan or other known mechanism for cooling an environment. The design and construction of these conventional features is a mere matter of design choice, and well within the abilities of those of ordinary skill in the art without undue experimentation.

A patient lying on support surface 16 is subject to the electromagnetic field generated by means 18, with concomitant regulation of temperature in this area, thereby subjecting the patient to natural thermal regulation and intensification of physiological metabolism processes, producing a distinct improvement in his physical condition. Depending upon the desired course of treatment, it is possible for patients to remain in the inventive device for extended periods, without suffering any harmful effects. The invention can be used for long-term hospital patients (in which case the inventive device could be used as a hospital bed), or for patients being treated at home, during their normal sleep cycle (in which case the inventive device could be used as a bed).

Where a patient is being treated for any appreciable period of time, it is presently preferred that the magnetic field be cycled on and off in two hour intervals to maintain the body in a constant state of flux, which is currently believed to be most efficacious in treatment of a patient, but this is not a necessary component of the treatment process. Longer or shorter cycling periods, including a constant “on” condition, may be employed, as desired.

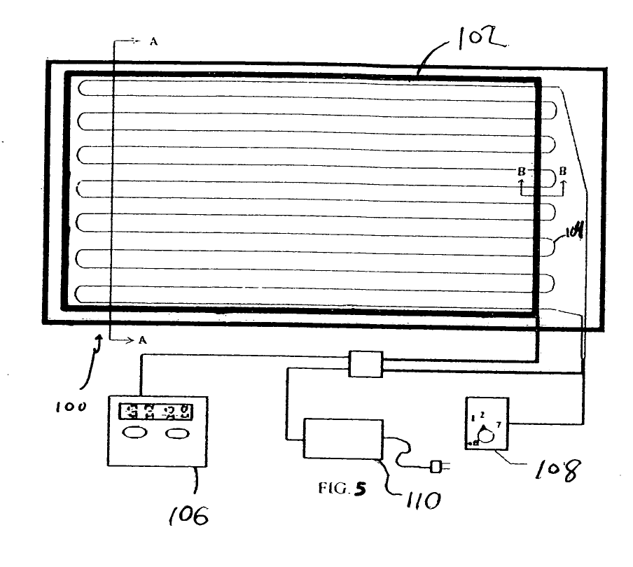

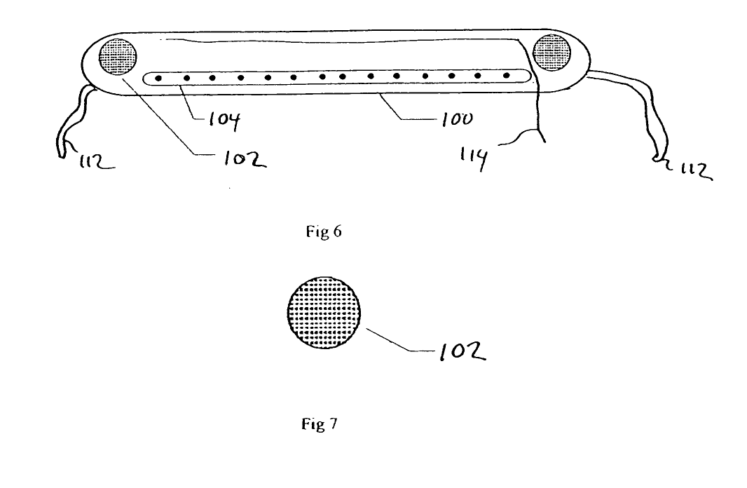

According to the invention herein, the device may be embodied as a flexible mattress pad, such as shown in FIG. 5, generally at 100. Mattress pad 100 is no more than six inches thick, preferably no more than two inches thick, and most preferably is in the range of from about one quarter inch to one inch thick. Mattress pad 100 includes inductor coils 102 to generate the electromagnetic field and heating coils 104 to warm the patient as desired. Inductor coils 102 may be controlled by a control box 106, and heating coils 104 may be controlled by a temperature control 108, which may be hand-held. Both control box 106 and temperature control 108 may be powered by a standard power supply 110.

Pad 100 may be shaped to conform to any size of mattress and may even be sized to provide the generated electromagnetic field and heating to less than the entire surface of the bed, for example, if the patient only requires treatment over a localized part of the body. Pad 100 may even be configured to be worn, and so may be sized to be positioned in an article of clothing.

Preferably, inductive coil 102 may be formed of 50 rounds of 28 gauge copper wire (FIG. 7).

Control box 106 may, in known fashion, be adapted to control the frequency of the generated electromagnetic field, the time the field is in operation and the intensity of the field.

Pad 100 may also include means 112 for attaching pad 100 to an object, such as a mattress or a user, if pad 100 is to be worn. Means 112 may be, for example, straps, resilient bands, snaps or other known equivalent means for attaching one object to another. One of ordinary skill in the art would be able to select a suitable means 12 for attaching pad 100 without undue experimentation, depending upon the desired application.

Pad 100 may also include a thread 114, similar to thread 30, woven into it, for removing electrostatic charge from the patient.

The invention is not limited by the embodiments described above which are presented as examples only but can be modified in various ways within the scope of protection defined by the appended patent claims.

For example, while the above description presupposes that the patient may be human, it should also be possible to treat other animal patients, particularly mammalian patients.

Thus, while there have been shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A portable device for treating a mammal, said mammal having a body with a magnetic field, comprising:
means for generating a magnetic field having an intensity of no more than about 2 mT;
means for maintaining said body of said mammal in said magnetic field;
means for regulating a body temperature for said mammal; and
a covering having a thickness of no more than about six inches, said covering housing at least one of said means for generating and said means for maintaining.

2. The device of claim 1, wherein said covering is flexible.

3. The device of claim 1, further comprising means for attaching said device to an object.

4. The device of claim 3, wherein said object is a bed.

5. The device of claim 3, wherein said object is the mammal.

6. The device of claim 1, wherein said means for regulating said body temperature of said mammal includes means for uniformly heating said body of said mammal, except for a head of said mammal.

7. The device of claim 6, wherein said means for heating said body of said mammal includes means for maintaining a body heat of said mammal at a predetermined temperature.

8. The device of claim 7, wherein said predetermined temperature is substantially in the range of from about 65° F. to about 100° F.

9. The device of claim 7, further comprising means for selecting said predetermined temperature.

10. The device of claim 1, wherein said covering houses both said means for generating and said means for maintaining.

11. The device of claim 1, further comprising means for removing electrostatic charge from said mammal present in said device

12. The device of claim 1, wherein said covering includes a fabric surface for disposition on the side of said covering facing said mammal.

13. The device of claim 12, wherein said fabric surface includes a wire positioned in said fabric surface to remove electrostatic charge from said patient.

14. The device of claim 1, wherein said magnetic field generating means comprises a plurality of inductive coils.

15. A portable device for treating a mammal, said mammal having a body with a magnetic field, comprising:
means for generating a magnetic field having an intensity of no more than about 2 mT;
means for maintaining said body of said mammal in said magnetic field;
means for regulating a body temperature for said mammal; and
a flexible covering having a thickness of no more than about six inches, said covering housing at least one of said means for generating, said means for maintaining and said means for regulating.

16. The device of claim 15, further comprising means for removing electrostatic charge from said mammal present in said device.

17. The device of claim 15, wherein said flexible covering includes a fabric surface for disposition on the side of said flexible covering facing said mammal.

18. The device of claim 15, wherein said thickness is no more than about two inches.

19. The device of claim 18, wherein said thickness is between about one half inch and about one inch
